(19) United States
(12) Patent Application Publication
(10) Pub. No.: US 2017/0010251 A1
(43) Pub. Date: Jan. 12, 2017

(54) Fluid Phase Analyzer with Embedded Measurement Electronics

(71) Applicant: Phase Dynamics, Inc., Richardson, TX (US)

(72) Inventor: Bentley N. SCOTT, Garland, TX (US)

(73) Assignee: Phase Dynamics, Inc., Richardson, TX (US)

(21) Appl. No.: 15/171,643

(22) Filed: Jun. 2, 2016

Related U.S. Application Data
(60) Provisional application No. 62/189,307, filed on Jul. 7, 2015.

(51) Int. Cl. G01N 33/28; G01N 22/04

(52) U.S. Cl. CPC G01N 33/2847; G01N 22/04; G01N 33/2823

(57) ABSTRACT

An apparatus for analyzing a multiphase fluid in a pipeline. The apparatus comprises: i) an elongated shaft adapted to be inserted into the pipeline, the elongated shaft comprising a measurement electronics section and an extension section; ii) a housing coupled to the elongated shaft and adapted to be positioned outside the pipeline when the elongated shaft is inserted into the pipeline; and iii) a ground cage coupled to the elongated shaft, the ground cage comprising a sensor coupled to the measurement electronics section. The ground cage comprises a tube having perforations therein to permit multiphase fluid to flow within the ground cage. The sensor comprises a ceramic rod and an antenna within the ceramic rod.

### CROSS-REFERENCE TO RELATED APPLICATION(S) AND CLAIM OF PRIORITY

The present application is related to U.S. Provisional Patent No. 62/189,307, entitled “Analyzer With Embedded Measurement Electronics”, and filed on Jul. 7, 2015. Provisional Patent No. 62/189,307 is assigned to the assignee of the present application and is hereby incorporated by reference into the present application as if fully set forth herein. The present application claims priority under 35 U.S.C. §119(e) to U.S. Provisional Patent No. 62/189,307.

### TECHNICAL FIELD

The present disclosure relates generally to apparatuses and methods for characterizing a multiphase fluid flow stream that has varying phase proportions over time and, in particular, to improved systems and methods for measuring the amount of oil, water, and gas in a pipeline.

### BACKGROUND

Crude petroleum oil and gaseous hydrocarbons are produced by extraction from subterranean reservoirs. In reservoirs with enough natural pressure, oil and gas flows to the surface without secondary lift techniques. Often, however, other methods are required to bring them to the surface. These include a variety of pumping, injection, and lifting techniques used at various locations, such as at the surface wellhead (e.g., rocking beam suction pumping), at the bottom of the well (e.g., submersed pumping), with gas injection into the well casing creating lift, and other techniques. Each technique results in oil and gas emerging from the well head as a multiphase fluid with varying proportions of oil, water, and gas. For example, a gas lift well has large volumes of gas associated with the well. The gas-to-oil volume ratios can be 200 cubic feet or more of gas per barrel. Large measurement uncertainties may occur, depending upon the methods used.

The measurement of water in petrochemical products is a common practice in the petroleum industry. This measurement is frequently done in combination with oil well testing to assist in optimizing oil production from a single oil well or a series of oil wells. The measurement may also be performed during the transfer of crude petroleum oil, as occurs during the production, transport, refining, and sale of oil. Specifically, it is well known to a person having ordinary skill in the art of petroleum engineering that crude petroleum oil emerging from production wells can contain large amounts of water, ranging from generally about 1% to as high as about 95% water. This value is known as the water cut (“WC”). Multiphase measurements typically provide an oil company and other stakeholders with the amount of gas, oil, and water and the average temperature, pressure, gas/oil ratio, and gas volume fraction that a well produces in a day.

Typical techniques to determine the water percentage or water cut is to use a capacitive, radio frequency, or microwave analyzer to perform the in-line monitoring of the oil and water mixture within a pipeline. U.S. Pat. No. 4,862,060 to Scott, entitled “Microwave Apparatus for Measuring Fluid Mixtures”, discloses microwave apparatuses and methods which are most suitable for monitoring water percentages when the water is dispersed in a continuous oil phase. U.S. Pat. No. 4,862,060 is hereby incorporated by reference as if fully set forth herein.

A conventional multiphase fluid analyzer typically comprises a sensor that is inserted into a pipeline through a flange. An electronics housing that is located outside the pipeline is connected to the sensor and measures signals from the sensor. However, the accuracy of such measurements are limited by complex influences, such as interfacial polarization at frequencies below 50 MHz, attenuation of the RF/microwave signals along sensor paths, the physical length with respect to a wavelength which causes multiples of a 180-degree phase shift, and temperature fluctuations of the multiphase fluids. Conventional multiphase fluid analyzers often minimized these problems by limiting the length of the measurement paths or the sensor and the frequency of measurement. Also, some conventional systems added temperature conditioning of the measurement electronics to control the ambient temperature effect on the measurement.

Thus, there is a need for improved systems and methods for measuring the water cut of a multiphase fluid. In particular, there is a need for a multiphase fluid analyzer capable of taking accurate water cut measurements across a wide spectrum of operating frequencies.

### SUMMARY

To address the above-discussed deficiencies of the prior art, it is a primary object to provide an apparatus for analyzing a multiphase fluid in a pipeline. In one embodiment, the apparatus comprises: i) an elongated shaft adapted to be inserted into the pipeline, the elongated shaft comprising a measurement electronics section and an extension section; ii) a housing coupled to the elongated shaft and adapted to be positioned outside the pipeline when the elongated shaft is inserted into the pipeline; and iii) a ground cage coupled to the elongated shaft, the ground cage comprising a sensor coupled to the measurement electronics section.

In one embodiment, the ground cage comprises a tube having perforations therein to permit multiphase fluid to flow within the ground cage.

In another embodiment, the sensor comprises a ceramic rod and an antenna within the ceramic rod.

In still another embodiment, the extension section may be varied in length to insert the sensor a desired distance into the multiphase fluid.

In yet another embodiment, the extension section comprises a data cable configured to transmit measurement data from the measurement electronics section to monitoring circuitry in the housing.

In a further embodiment, the housing is coupled to the pipeline by means of a first liquid tight seal.

In a still further embodiment, the extension section is coupled to the measurement electronics section my means of a second liquid tight seal.

In a yet further embodiment, the measurement electronics section sensor comprises a circuit board configured to provide at least a radio frequency (RF) signal to the sensor.

In one embodiment, the circuit board comprises a temperature sensing element operable to sense a temperature of the multiphase fluid.

In another embodiment, the circuit board comprises measurement circuitry coupled to the sensor that adapts measured data measured by the sensor according to the temperature sensed by the temperature sensing element.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely. Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates a fluid phase analyzer according to an embodiment of the present disclosure.

FIG. 2 illustrates the fluid phase analyzer of FIG. 1 in greater detail according to an embodiment of the present disclosure.

FIG. 3 illustrates selected portions of the fluid phase analyzer of FIG. 1 in greater detail according to an embodiment of the present disclosure.

FIG. 4 illustrates selected portions of the fluid phase analyzer of FIG. 1 in greater detail according to an embodiment of the present disclosure.

FIG. 5 illustrates a plurality of the fluid phase analyzers used to analyze multiphase fluids in a reservoir according to an embodiment of the present disclosure.

### DETAILED DESCRIPTION

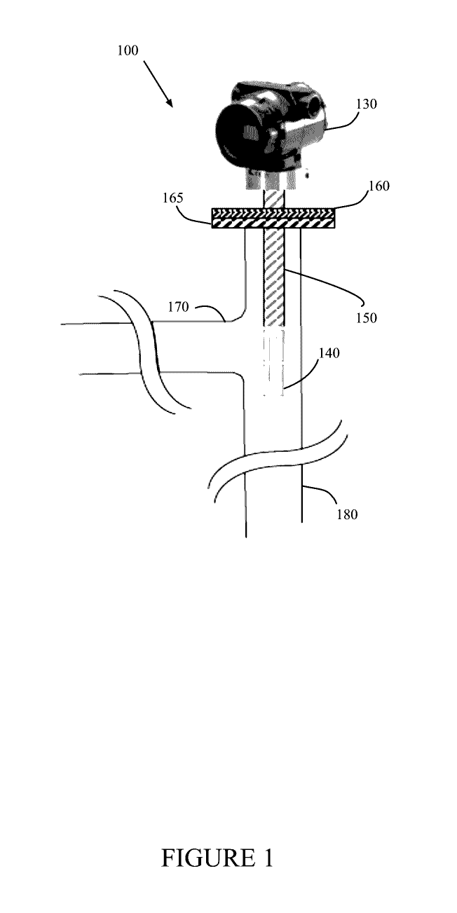

FIGS. 1 through 5, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged petroleum production pipeline infrastructure.

The present disclosure generally relates to systems and methods for measuring the amount of one phase in a mixture of phases and, more particularly, to measuring the amount of water present in crude petroleum oil. This disclosure describes an apparatus in which the measurement electronics are embedded in the shaft of the analyzer that is inserted into the multiphase fluid. This system configuration reduces the parasitic length found in the prior art from affecting the measurement, thereby providing more accurate and reproducible measurements. This configuration also improves the ability to measure at higher frequencies, thereby providing increased resolution of measurement. In the prior art phase analyzers, the added length of the waveguide would be detrimental due to the radio frequency (RF) losses and phase lengths involved.

Some embodiments of the disclosed apparatus are methods and systems for determining the amount of water in crude petroleum oil. As crude petroleum oil is held over time, gravitationally-induced separation of water-continuous and oil-continuous phases can occur. At least some of the properties of the separated phases can be used to generate water and oil property values which in turn can be used to provide improved water percentage determinations of crude petroleum oil.

Some embodiments of the disclosed apparatus are used to determine the water fraction and the oil fraction in an oil and water mixture which has been subjected to gravity and un-agitated storage. For example, the disclosed apparatus may be used to sample, measure, and analyze petroleum being off-loaded from a transport tanker, in which some gravitationally-induced phase separation of a water-continuous phase and an oil-continuous phase has occurred in the hold during transit. Also, the disclosed apparatus may be used to measure and to characterize crude petroleum oils being pumped from a storage vessel, in which some gravitationally-induced phase separation of a water-continuous phase and an oil-continuous phase has occurred in the tank during storage. Some embodiments of the disclosed apparatus are used to determine the level in a stored oil tank. This is especially used during water draw from the bottom of the tank to determine when to stop the water flow.

The disclosed innovations, in various embodiments, provide one or more of at least the following advantages: i) some of the measurement electronics are moved down to the measurement area to improve the confidence level in determining the amount of water in crude petroleum oil; ii) improved measurement due to reduction of the attenuation between the signal source and the measurement area; iii) a reduction of the phase length of the signal between the signal source and the measurement area; iv) compensation for the ambient change of temperature with respect to the operating point of the measurement electronics using a temperature sensing element; and v) real-time reduction of errors and supplying more accurate results, thereby aiding near-real-time decision-making or automatic flow diversion, without requiring oil stream sampling or off-line lab-work, thereby reducing cost, lost opportunities, and hazards associated with such sampling.

FIG. 1 illustrates fluid phase analyzer 100 according to an embodiment of the present disclosure. Fluid phase analyzer 100 comprises electronics housing 130, ground cage 140, variable-length extension shaft 150, and flange 160. Ground cage 140 and extension shaft 150 are inserted through flange 165 into a “T-shaped” pipe section comprising pipeline 170 and pipeline 180. Flange 165 is welded to the T-shaped pipe section. Extension shaft 150 may be welded through a hole in flange 160 so that when flange 160 is bolted or welded onto flange 165, a fluid-tight seal is created. However, electronics housing 130 remain outside of the T-shaped pipeline in the ambient air temperature, while only extension shaft 150 and ground cage 140 are immersed in the multiphase fluid inside of the T-shaped pipe section.

In an exemplary embodiment, ground cage 140 comprises a coaxial shaft with a ceramic center rod, wherein an antenna is disposed inside of the ceramic rod. The ceramic rod allows RF wave propagation through water continuous (conductive) emulsions and is thick enough to allow electrical propagation while establishing the current (magnetic) propagation through the conductive medium as described in U.S. Pat. No. 4,862,060, incorporated by reference above. In an exemplary embodiment, one or both of ground cage 140 and extension shaft 150 may be metal tubes that are cylindrical in shape (i.e., circular cross-sectional area). However, in alternate embodiments, one or both of ground cage 140 and extension shaft 150 may have a differently shaped cross-sectional area, including oval, triangular, rectangular, and the like.

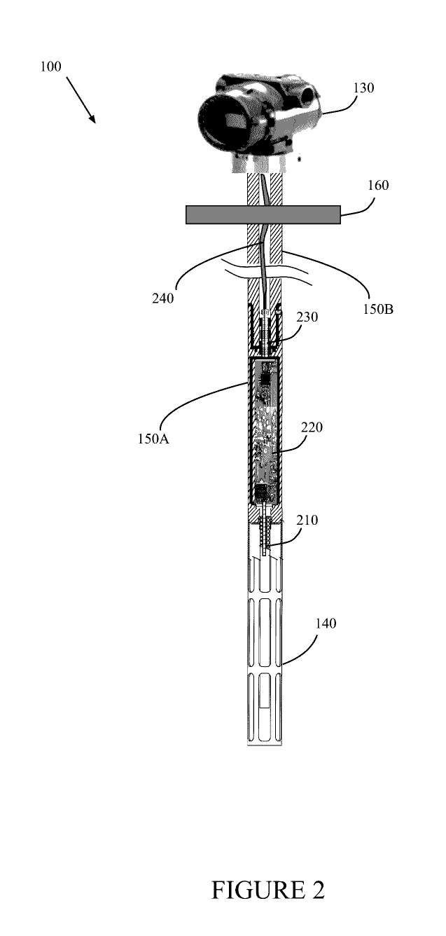

FIG. 2 illustrates fluid phase analyzer 100 in greater detail according to an embodiment of the present disclosure. In the exemplary embodiment, extension shaft 150 actually comprises two sections: measurement electronics section 150A and extension section 150B. Measurement electronics section 150A is threaded onto extension section 150B. The length of extension section 150B varies according to how deep the sensor in ground cage 140 must be inserted into a multiphase fluid in a particular implementation.

Measurement electronics section 150A comprises circuit board 220 (shown in a top view), which is coupled at one end to sensor 210 in ground cage 140. As noted above, sensor 220 comprises a ceramic center rod, wherein a coaxial antenna is disposed inside of the ceramic rod. Measurement electronics section 150A is coupled at the other end by connector 230 to cable 240. Cable 240 is, in turn, coupled to, for example, a microcontroller and a transceiver inside electronics housing 130. Cable 140 may comprise, among others, a power line, a ground line, and a twisted pair signal line for communicating with the circuitry inside electronics housing 130.

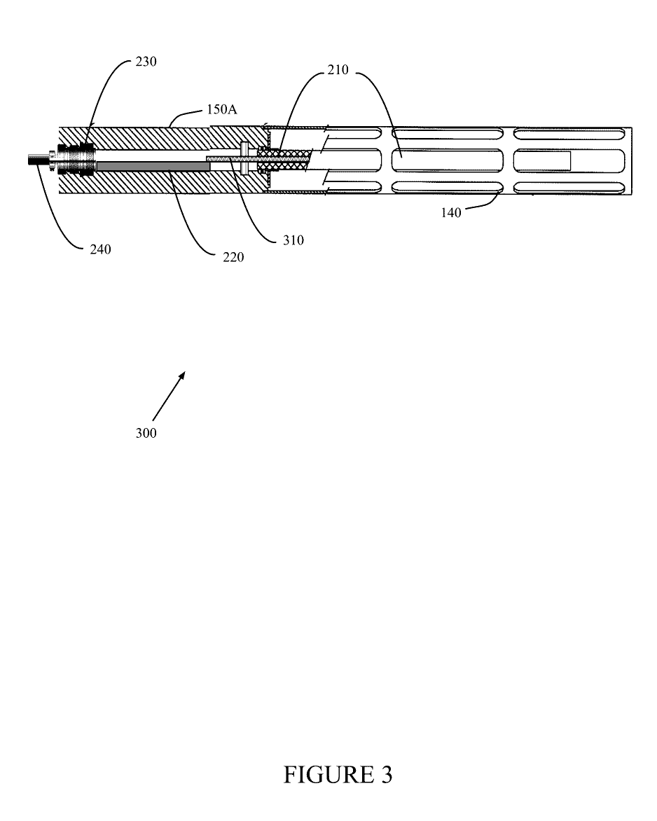

FIG. 3 illustrates selected portions of fluid phase analyzer 100 in greater detail according to an embodiment of the present disclosure. FIG. 3 provides a side view of circuit board 220. Antenna 310 is coupled to circuitry on circuit board 220 and is inserted into the ceramic body of sensor 210, which extends into ground cage 140. More generally, sensor 210 may comprise any antenna structure that provide electromagnetic propagation and may include non-ceramic materials.

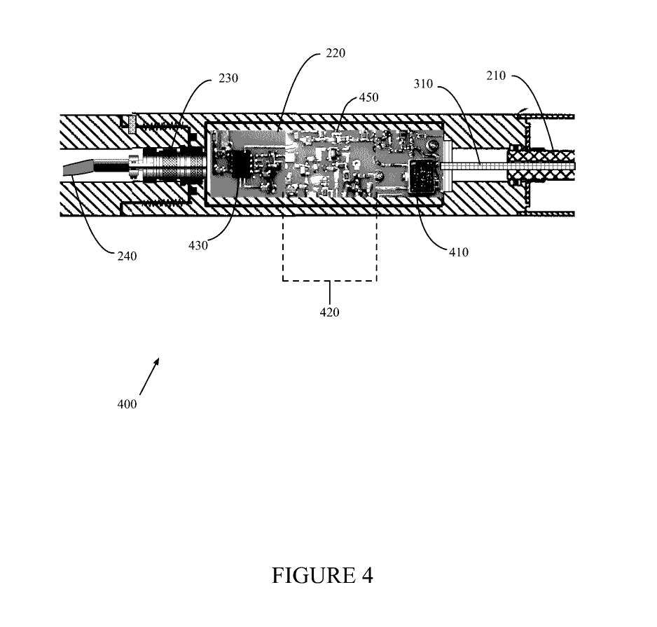

FIG. 4 illustrates selected portions of fluid phase analyzer 100 in greater detail according to an embodiment of the present disclosure. As shown in FIG. 4, circuit board 220 comprises radio frequency (RF) transceiver circuitry 410, sampling and measurement circuitry 420, input-output (I/O) interface circuitry 430, and temperature sensing element 450. More generally, temperature sensing element 450 may comprise any element capable of measuring the apparent fluid temperature, such as a resistive temperature device (RTD), in order to compensate for the variations in the RF/microwave properties of the oil and water. For example, an Analog Devices AD592 may be used to measure temperature. RF transceiver circuitry 410 drives coaxial antenna 310 with an RF signal and receives from antenna 310 reflected RF signals. Sampling and measurement circuitry 420 measures the reflected signals received from antenna 310 to determine power measurements, phase detection, and/or load pull measurement. I/O interface circuitry communicates with sampling and measurement circuitry 420 and the circuitry in electronics housing 130 to relay measurement data to electronics housing 130 and receive command signals and configuration data from electronics housing 130. Temperature sensing element 450 provides compensation for local temperature and local temperature measurement.

By way of example, in accordance with the apparatus disclosed in column 4 of U.S. Pat. No. 4,996,490, sampling and measurement circuitry 420 may comprise a microwave or radio frequency range signal generator connected to antenna 310 for generating a high frequency signal which may be varied by a voltage controlled oscillator tuning circuit. A signal receiver monitors the change in frequency caused by impedance pulling of the oscillator due to the change in fluid dielectric constant and transmits a differential frequency signal to a frequency counter and microprocessor for comparison of the measured signal with known reference signals for determining the percentage of water and oil in the multiphase fluid.

Measurement electronics section 150A is sealed in two places—by the ceramic-to-metal seal formed by sensor 210 at one end and by the welded connector 230 at the other end. Extension section 150B attaches to measurement electronics section 150 on one end and to electronic housing 130 on the other end and may be of any length and flange type at the process connection. The threads connecting measurement electronics section 150A and extension section 150B are O-ring sealed and may be locked into position with Allen screws or other methods to capture the two pieces. Extension section 150B may be made smaller than measurement electronics section 150A for convenient installation since extension section 150B only needs to be capable of withstanding the process and flange pressures and stresses. Measurement electronics section 150A becomes a totally sealed unit capable of operation in the severe oilfield environment. In addition, the circuitry may be intrinsically safe to prevent any potential hazard from occurring if the process seal is compromised.

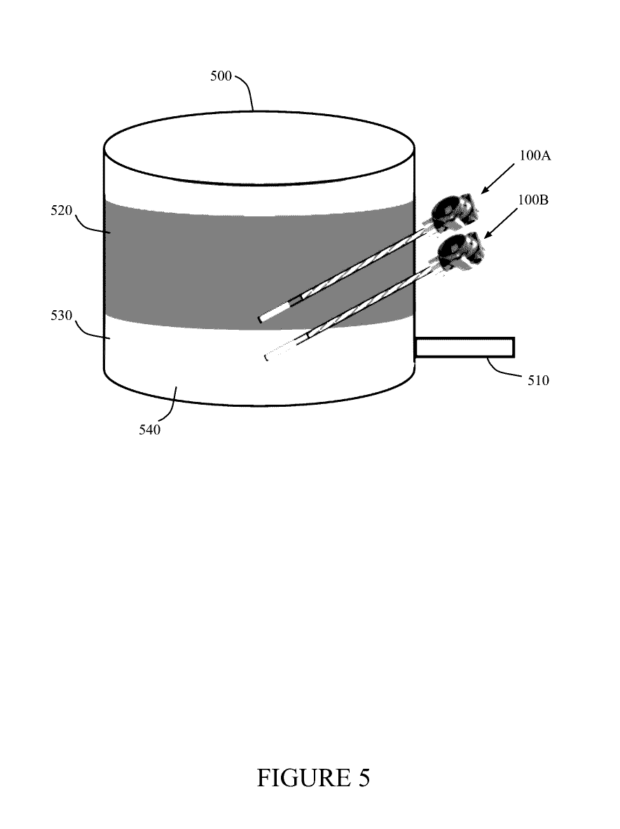

FIG. 5 illustrates a plurality of fluid phase analyzers 100A and 100B being used to analyze multiphase fluids in a reservoir according to an embodiment of the present disclosure. Within petroleum tank 500, oil layer 520 is separated from water layer 540 by emulsion layer 530. Outlet pipe 510 draws free water off the bottom of tank 500. FIG. 5 shows the measurement electronic sections of fluid phase analyzers 100A and 100B deep within petroleum tank 500. This is accomplished by using very long extension sections 150. This embodiment uses two fluid phase analyzers 100A and 100B to indicate when the interface (i.e., emulsion layer 530) between oil layer 520 and water layer 540 comes past the sensors in order to shut the draw valve (not shown) on outlet pipe 510 before oil is delivered to the water clean-up facility. If the oil content is too high (typically more than 5%), this may clog the floatation cells.

Existing capacitance interface probes are not capable of making measurements at high water content when the emulsion is in the water continuous emulsion phase. Prior art devices will measure 100% water when the emulsion is oil continuous and high in water content (75% and above depending upon the oil). These high water, oil continuous emulsions are sometimes called “rag layers” and may be from several inches to several feet thick. These do not separate with time but require heat and chemical emulsion breakers. As a result, the rag layer may be delivered to the pipeline which should be almost clean water. If the “rag layer” was pumped to the water cleanup facility it would potentially create difficult problems at that facility.

There are no probes that exist today that can both detect this emulsion phase at high water percentages (without calling it 100%) and make an accurate measurement of the water content. This is because the prior art devices are capacitance probes which short-out electrically in this emulsion. Conventional RF/microwave systems are unable to make an accurate measurement because the length of the probe is too long, which causes attenuation and phase length problems. However, improved fluid phase analyzers 100 according to the principles of the present disclosure are capable of such measurements because the measurement electronics are moved out of housing 130 and down into the probe that is immersed in the multi-phase fluid.

Although the present disclosure has been described with an exemplary embodiment, various changes and modifications may be suggested to one skilled in the art. It is intended that the present disclosure encompass such changes and modifications as fall within the scope of the appended claims.

What is claimed is:

1. An apparatus for analyzing a multiphase fluid in a pipeline comprising:
an elongated shaft adapted to be inserted into the pipeline, the elongated shaft comprising a measurement electronics section and an extension section;
a housing coupled to the elongated shaft and adapted to be positioned outside the pipeline when the elongated shaft is inserted into the pipeline; and
a ground cage coupled to the elongated shaft, the ground cage comprising a sensor coupled to the measurement electronics section.

2. The apparatus as set forth in claim 1, wherein the ground cage comprises a tube having perforations therein to permit multiphase fluid to flow within the ground cage.

3. The apparatus as set forth in claim 1, wherein the sensor comprises a ceramic rod and an antenna within the ceramic rod.

4. The apparatus as set forth in claim 1, wherein the extension section may be varied in length to insert the sensor a desired distance into the multiphase fluid.

5. The apparatus as set forth in claim 1, wherein the extension section comprises a data cable configured to transmit measurement data from the measurement electronics section to monitoring circuitry in the housing.

6. The apparatus as set forth in claim 1, wherein the housing is coupled to the pipeline by means of a first liquid tight seal.

7. The apparatus as set forth in claim 6, wherein the extension section is coupled to the measurement electronics section my means of a second liquid tight seal.

8. The apparatus as set forth in claim 1, wherein the measurement electronics section sensor comprises a circuit board configured to provide at least a radio frequency (RF) signal to the sensor.

9. The apparatus as set forth in claim 8, wherein the circuit board comprises a temperature sensing element operable to sense a temperature of the multiphase fluid.

10. The apparatus as set forth in claim 9, wherein the circuit board comprises measurement circuitry coupled to the sensor that adapts measured data measured by the sensor according to the temperature sensed by the temperature sensing element.
